(19) United States
(12) Patent Application Publication
(10) Pub. No.: US 2026/0021217 A1
(43) Pub. Date: Jan. 22, 2026

(54) SMART LIVESTOCK FACILITY SYSTEM CAPABLE OF ACTIVE DISEASE PREDICTION AND CONTROL BASED ON DATA-DRIVEN INTELLIGENT SPRAYING AND AIR CONDITIONING TECHNOLOGY

(71) Applicant: HANSOL ROOTONE AGRICULTURAL CORPORATION CO., LTD., Dangjin-si Chungcheongnam-do (KR)

(72) Inventors: Hansol HWANG, Dangjin-si Chungcheongnam-do (KR); Hyungkook KIM, Seongnam-si Gyeonggi-do (KR)

(73) Assignee: HANSOL ROOTONE AGRICULTURAL CORPORATION CO., LTD., Dangjin-si Chungcheongnam-do (KR)

(21) Appl. No.: 19/345,317

(22) Filed: Sep. 30, 2025

Related U.S. Application Data
(63) Continuation of application No. PCT/KR2024/003519, filed on Mar. 20, 2024.

(30) Foreign Application Priority Data
Feb. 23, 2024 (KR) 10-2024-0026508

(51) Int. Cl. A61L 9/14; A01K 29/00

(52) U.S. Cl. CPC A61L 9/145; A01K 29/005; A61L 2209/111; A61L 2209/14

(57) ABSTRACT

According to one embodiment of the present invention, the smart livestock housing system capable of active disease prediction and control based on data-driven intelligent spraying and air conditioning technology is specifically a technology designed to prevent predictable diseases and actively control quarantine systems within farms to prevent animal infectious diseases and ultimately improve farm productivity. The smart livestock housing system may comprise: a housing for raising animals; a plurality of opening/closing ports formed on one side and another side of the housing, respectively, to control inflow of outside air into the interior; a breeding space installed inside the housing and formed to bound the space where animals are raised in all directions; at least one filter formed in one area constituting the breeding space; and an injector located between the opening/closing ports and the filter, operating to remove viruses by spraying disinfectant into outside air entering from the opening/closing ports.

### CROSS-REFERENCE TO RELATED APPLICATIONS

This application is a continuation of International Patent Application No. PCT/KR2024/003519 filed on Mar. 20, 2024, which claims priority to and the benefit of Korean Patent Application No. 10-2024-0026508 filed in the Korean Intellectual Property Office on Feb. 23, 2024, the entire contents of which are incorporated herein by reference.

### 1. FIELD

The present invention relates to a smart livestock housing system capable of active disease prediction and control based on data-driven intelligent spraying and air conditioning technology. This technology aims to solve fundamental problems of existing inspection methods for highly pathogenic animal diseases and existing livestock housing structures that cannot prevent such diseases through the introduction of big data and smart livestock housing systems. Specifically, this technology is designed to prevent predictable diseases and actively control quarantine systems within farms to prevent animal infectious diseases and ultimately improve farm productivity.

### 2. DESCRIPTION OF THE RELATED ART

Recently, “Smart Farm” systems that integrate Information and Communications Technology (ICT) into agriculture have been distributed to farms under government leadership, enabling observation, control, and management of farming environments in remote and automated settings. While various ICT and IoT technologies are being developed and distributed in the livestock sector, except for equipment modernization, no significant results have been achieved. In particular, the current livestock systems and structures are vulnerable to fatal highly pathogenic livestock infectious diseases such as avian influenza (AI), foot-and-mouth disease (FMD), and African swine fever (ASF), which frequently occur due to abnormal climate conditions. The development of systems capable of predicting and controlling these diseases is an urgent task.

Furthermore, current devices capable of determining the presence of highly pathogenic diseases include periodic precision inspections during vulnerable periods, PCR testing for infection upon discovery of symptoms such as mass mortality, and precision testing that requires up to 48 hours when positive results are obtained. This structure is not suitable for infectious disease response manuals that require urgency.

Therefore, the development of a disease monitoring system (A Surveillance Farming System to Monitor Symptoms and React Immediately to the Severe Pandemics) that enables producers to actively respond to diseases on-site using ultra-high speed, ultra-low latency, and ultra-connected broadband wireless communication network (5G) technology is essential.

However, most livestock housing systems still cannot fundamentally block sensitive diseases such as avian influenza when they occur in poultry, and furthermore, even avian influenza prediction or response systems fail to provide appropriate countermeasures. For example, FIG. 1 is a schematic diagram of a conventional livestock housing system, where filters and fans are installed in the livestock housing to build a system for filtering viruses entering from outside. However, only simple filters are installed, and due to performance issues with these filters, some viruses still penetrate into the livestock housing, failing to significantly help prevent disease infection in poultry.

### SUMMARY

The present invention aims to solve the aforementioned problems of conventional animal infectious disease prediction and diagnosis technologies by utilizing ICT and IoT technologies to provide a smart livestock housing system capable of active diagnosis and treatment of virus penetration through smart virus detection sensors, artificial intelligence-based disinfection spraying device systems, and virus penetration assessment capabilities.

Additionally, the present invention aims to provide a spraying device capable of targeting and immediately destroying viruses to achieve fundamental blocking of sensitive disease infections such as avian influenza.

The problems to be solved by the present invention are not limited to the problems mentioned above, and other problems not mentioned will be clearly understood from the description below.

According to one embodiment of the present invention, an active smart livestock housing system capable of disease prevention comprises: a housing formed to have an internal space for raising animals; a plurality of opening/closing ports formed on one side and another side of the housing, respectively, to control inflow of outside air into the interior; a breeding space installed inside the housing and formed to bound the space where animals are raised in all directions; at least one filter formed in one area constituting the breeding space; and an injector located between the opening/closing ports and the filter, operating to remove viruses by spraying disinfectant into outside air entering from the opening/closing ports.

Additionally, the system further comprises a first sensor unit installed on an outer wall of the housing and a second sensor unit installed inside the housing, wherein the first sensor unit and the second sensor unit each measure at least one of temperature, humidity, carbon dioxide concentration, and virus detection presence, and when the second sensor unit is installed inside the breeding space, a plurality of the second sensor units may be installed on the floor, sides, and ceiling.

Additionally, the first sensor unit and the second sensor unit may be configured in the form of a substrate combined with aptamers and reaction solutions, configured to change color when viruses combine with the aptamers and reaction solutions.

Additionally, the system further comprises a heating/cooling air conditioning device disposed between the at least one opening/closing port and the filter, wherein the heating/cooling air conditioning device may perform cooling or heating operations when outside air is introduced into the interior, controlling at least one of temperature and humidity of air introduced into the breeding space.

Additionally, the system further comprises a fan installed on an inner side of the housing adjacent to the opening/closing ports, wherein the fan may be configured as an intake fan or an exhaust fan to control a speed at which outside air flows in or out.

Additionally, the breeding space is configured to be sealed except for the space where the filter is installed and is configured to be spaced apart from the opening/closing ports, wherein the space between the breeding space and the opening/closing ports may include at least one of a first separation space and a second separation space, and the number of separation spaces at the position where outside air is introduced may differ from the number of separation spaces at the position where outside air is discharged.

Additionally, the first separation space and the second separation space are separated by sealed walls, wherein outside air is introduced into the first separation space, cooled or heated outside air is introduced into the second separation space by the heating/cooling air conditioning device between the first separation space and the second separation space, and viruses in outside air introduced into the second separation space may be removed by disinfectant sprayed from the filter and the injector.

Additionally, the system further comprises a sensor installed on the filter to detect presence or absence of viruses, wherein when the sensor detects viruses, the injector may operate to target a direction in which the sensor is located and then perform spraying operations to destroy the viruses.

Additionally, each opening/closing port is installed on different surfaces spaced apart from each other within the housing to discharge outside air introduced from one opening/closing port, and the filter and the injector are located adjacent to each opening/closing port to perform virus removal functions.

Additionally, the system may further comprise a server that monitors status of devices including the first sensor unit and the second sensor unit, opening/closing ports, heating/cooling air conditioning device, injector, and fan, and controls operation of the devices.

According to another embodiment of the present invention, a method for operating an active smart livestock housing system capable of disease prevention comprises: operating a plurality of opening/closing ports formed on one side and another side of a housing formed to have an internal space for raising animals, respectively, to control inflow of outside air, thereby introducing outside air into the interior of the housing; performing an operation to remove viruses by spraying disinfectant into outside air through an injector; and allowing outside air that has passed through the injector to be introduced into an interior of a breeding space through a filter, wherein the breeding space is formed to bound the space where animals are raised in all directions

According to one embodiment of the present invention, the smart livestock housing system can control IoT devices within the livestock housing system according to external disease occurrence status or external virus occurrence, thereby making the environment comfortable for animals in the livestock housing and preventing disease occurrence.

Furthermore, as the server controls the system with IoT devices based on overall regional disease occurrence status, it can actively control the system and prevent disease infection in animals.

### BRIEF DESCRIPTION OF THE DRAWINGS

FIG. 1 is a diagram illustrating problems of a conventional livestock housing system.

FIG. 2 is a structural diagram of an entire system according to one embodiment of the present invention.

FIG. 3 is a block diagram of a server structure according to one embodiment of the present invention.

FIG. 4 is a schematic diagram of an active smart livestock housing system according to one embodiment of the present invention.

FIG. 5 is a schematic diagram of an active smart livestock housing system according to one embodiment of the present invention, showing operation in outside air circulation mode.

FIG. 6 is a diagram showing an example of a user interface provided to a user terminal of an active smart livestock housing system according to one embodiment of the present invention.

FIG. 7 is a flowchart of a driving method for an active smart livestock housing system according to one embodiment of the present invention.

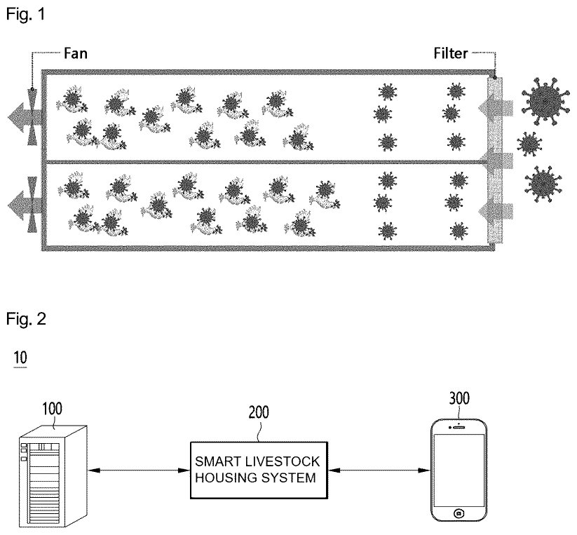

### DETAILED DESCRIPTION

Hereinafter, embodiments of the present disclosure will be described in detail such that those skilled in the art to which the present disclosure belongs may easily implement the present disclosure with reference to the accompanying drawings. However, the present disclosure may be implemented in many different forms and is not limited to the embodiments to be described herein. In addition, in order to clearly describe the present disclosure with reference to the drawings, portions irrelevant to the description are omitted, and similar reference numerals are attached to similar portions throughout the specification.

Throughout the present specification, when a portion is described to be “connected” to another portion, this includes not only a case where the portion is “directly connected” thereto, but also a case where the portion is “electrically connected” thereto with another element therebetween. In addition, when a certain portion is described to “include” a certain component, this means that the certain portion may further include other components without excluding other components unless otherwise stated.

In the present disclosure, a “portion” includes a unit realized by hardware, a unit realized by software, and a unit realized by using both. In addition, one unit may be realized by using two or more pieces of hardware, and two or more units may be realized by using one piece of hardware. Meanwhile, a “˜portion” is not limited to software or hardware, and a “˜portion” may be configured to be included in an addressable storage medium or may be configured to reproduce one or more processors. Therefore, in one example, “˜portion” refers to components, such as software components, object-oriented software components, class components, and task components, and includes processes, functions, properties, procedures, subroutines, segments of program code, drivers, firmware, microcode, circuits, data, databases, data structures, tables, arrays, and variables. The functions provided within the components and “˜portions” may be combined into a smaller number of components and “˜portions” or may be further separated into additional components and “˜portions”. Additionally, components and “˜portions” may be implemented to reproduce one or more processors or CPUs within a device or a security multimedia card.

A “terminal” to be described below may be implemented by a computer or a portable terminal capable of accessing a server or another terminal through a network. Here, the computer may include, for example, a notebook computer in which a web browser is stored, a desktop computer, a laptop computer, a Virtual Reality Head Mounted Display (VR HMD) (for example, HTC VIVE, Oculus Rift, GearVR, DayDream, PSVR, and so on), and so on. Here, the VR HMD includes a VR HMD for a Personal Computer (PC) (for example, HTC VIVE, Oculus Rift, FOVE, Deepon, and so on) and a VR HMD for a mobile terminal (for example, GearVR, DayDream, Stormtrooper, Google Cardboard, and so on), a standalone model that are independently implemented of VR HMD for a console (PSVR) (for example, Deepon, PICO, and so on), and so on. The portable terminal is, for example, a wireless communication device that ensures portability and mobility, and includes not only a smartphone, a tablet PC, and a wearable device, but also various devices equipped with communication modules, such as a Bluetooth (Bluetooth Low Energy (BLE)) module, a Near Field Communication (NFC) module, a Radio Frequency IDentification (RFID) module, and an ultrasonic module, an infrared module, a Wi-Fi module, and a LiFi module. In addition, the “network” refers to a connection structure capable of exchanging information between nodes, such as terminals and servers, and includes a Local Area Network (LAN), a Wide Area Network (WAN), the Internet (WWW: World Wide Web), a wired and wireless data communication network, a telephone network, a wired and wireless television communication network, and so on. For example, the wireless data communication network includes third generation (3G), fourth generation (4G), fifth generation (5G), third generation partnership project (3GPP), Long Term Evolution (LTE), World Interoperability for Microwave Access (WIMAX), Wi-Fi, Bluetooth communication, infrared communication, ultrasonic communication, Visible Light Communication (VLC), LiFi, and so on, but are not limited thereto.

Referring to FIG. 2, the entire system 10 according to one embodiment of the present invention may include a server 100, a smart livestock housing system 200, and a user terminal 300.

The server 100 may collect status information from IoT devices constituting the smart livestock housing system 200, monitor the smart livestock housing system 200, and control IoT devices to prevent virus entry into the livestock housing and manage the environment within the livestock housing cleanly to perform overall control functions for preventing disease infection in advance.

Furthermore, as an additional embodiment, the server 100 may also perform the role of predicting disease occurrence by collecting data from other livestock housing systems 200 or environmental data regarding temperature, humidity, etc. from external servers.

Here, IoT devices may include various sensor units 270, filters 260, injectors 250 for spraying disinfectants, air conditioning devices 240, etc.

The smart livestock housing system 200 includes not only the overall frame or housing 210 structure constituting the livestock housing, but also various sensor units 270 that collect status information of the livestock housing, and filters 260, injectors 250, air conditioning devices 240, opening/closing ports 220a, 220b, etc. that can prevent viruses from entering the livestock housing and manage the environment of the livestock housing cleanly.

The smart livestock housing system 200 can collect status information and control the internal environment based on IoT devices, thereby actively performing disease infection management.

The user terminal 300 may be a terminal of an administrator managing the smart livestock housing system 200. The user terminal 300 can remotely manage, supervise, and monitor the environment of the smart livestock housing system 200, and may directly input control commands as needed.

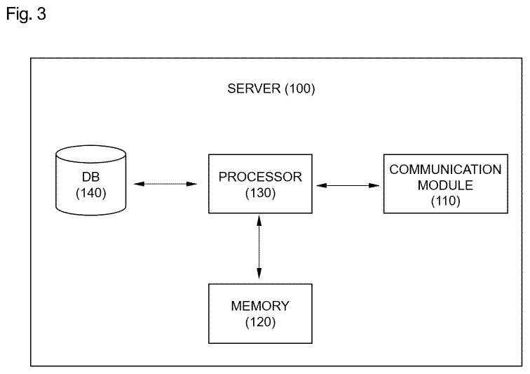

Referring to FIG. 3, the server 100 according to one embodiment of the present invention will be described in detail.

The server 100 according to one embodiment of the present invention may comprise a communication module 110, memory 120, processor 130, and DB 140.

The communication module 110 performs data transmission and reception with IoT devices constituting the smart livestock housing system 200 or data transmission and reception with external servers. Data transmitted and received from IoT devices may be data regarding the status of the smart livestock housing system 200. Data received from external servers varies in type and may include meteorological agency data, other livestock housing system 200 data, various news data, data on major diseases, etc.

The memory 120 may have programs installed and stored for performing methods of managing and controlling the smart livestock housing system 200.

The processor 130 may perform the role of monitoring, controlling, and managing the smart livestock housing system 200 by executing the above programs. The processor 130 may decide whether to operate the smart livestock housing system 200 in outside air circulation mode or inside air circulation mode as needed, and may also decide under what conditions to control the internal environment of the smart livestock housing system 200 within each mode. Prior to such decisions, the processor 130 may precede judgment on whether the environment inside the livestock housing is comfortable and whether virus penetration is expected by referring to the state of poultry according to status information of the smart livestock housing system 200.

The DB may store status information of the smart livestock housing system 200 received by the communication module or various data received from external servers.

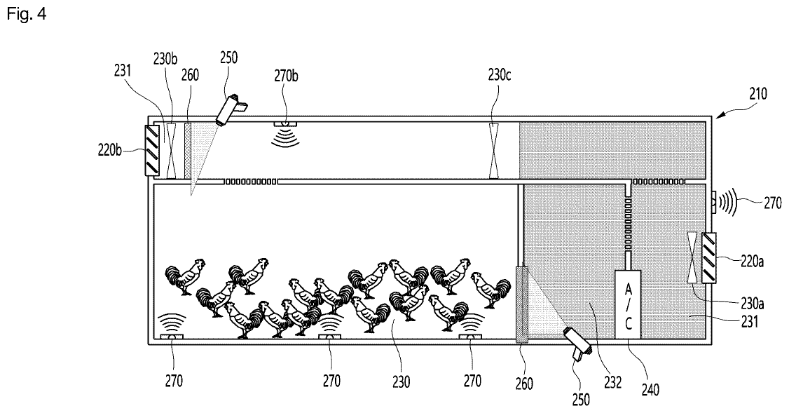

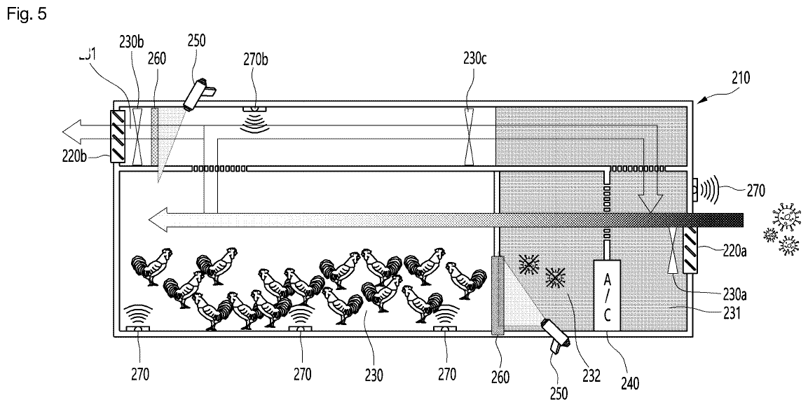

Hereinafter, referring to FIGS. 4 and 5, the smart livestock housing system 200 according to one embodiment of the present invention will be described in detail.

The smart livestock housing system 200 according to one embodiment of the present invention may include a housing 210, opening/closing ports 220a, 220b, fans 230a˜230c, breeding space 230, heating/cooling air conditioning device 240, injector 250, filter 260, and sensor units 270.

The housing 210 may refer to a structure composed of frames or skeletons, exterior materials, interior materials, etc. formed to have an internal space for raising animals (poultry).

The opening/closing ports 220a, 220b may be formed in plurality at various locations on the outer wall of the housing 210. The opening/closing ports 220a, 220b are formed on one side and another side of the housing 210, respectively, to control inflow of outside air into the interior. In the drawing, two opening/closing ports (first opening/closing port 220a and second opening/closing port 220b) are shown installed in completely opposite directions. This is to allow outside air introduced during outside air circulation to be easily discharged to the outside. However, this is only an example, and the opening/closing ports 220a, 220b do not necessarily need to be arranged in opposite directions, may be arranged at various different positions, and the installation height is also not limited to one example. Additionally, the opening/closing ports 220a, 220b include angle adjustment functions to adjust the angle at which outside air flows in or out.

Fans 230a-230c are installed on the inner side of the housing 210, that is, toward the inside of the livestock housing, to allow outside air flowing in or out through the opening/closing ports 220a, 220b to blow in or out more easily. Fans 230a-230c may be configured as either intake fans or exhaust fans depending on the role of opening/closing ports 220a, 220b. For example, referring to FIG. 5, outside air is introduced through the first opening/closing port 220a, passes through the first fan 230a and flows into the livestock housing interior, passes through the breeding space 230, and moves toward the second fan 230b or circulates back into the livestock housing interior, and outside air directed toward the second fan 230b exits outside through the second opening/closing port 220b. Outside air moved in the direction of circulating back into the livestock housing interior is directed to the first separation space 231 through the third fan 230c. At this time, the first fan 230a may be configured as an intake fan, and the second fan 230b and third fan 230c may be configured as exhaust fans.

The heating/cooling air conditioning device 240 can perform cooling or heating control. It collects temperature from temperature sensors (not shown) installed at each location within the livestock housing and, under control of the server 100, the heating/cooling air conditioning device 240 can control temperature or humidity within the livestock housing by setting appropriate temperatures and blowing cold air or warm air. By performing such heating and cooling control on outside air introduced through opening/closing ports 220a, 220b and fans 230a˜230c, temperature control of air within the livestock housing can be performed more smoothly.

The injector 250 may perform the role of spraying disinfectants capable of destroying viruses. The injector 250 can spray disinfectants because viruses may exist in outside air introduced through opening/closing ports 220a, 220b. For this purpose, it may be configured to include a container for storing disinfectants and spray nozzles. The direction in which the injector 250 is installed and sprays nozzles may be any direction, but may be installed in a direction toward the livestock housing interior or filter 260. Since the filter 260, described later, is installed on the only pathway through which air can enter the breeding space 230, the injector 250 may be installed in the direction of the filter 260 to prevent viruses from penetrating into the breeding space 230. The number and position of nozzles constituting the injector 250 may also be configured to enable spraying of disinfectants across the entire front surface of the filter 260.

Additionally, since virus penetration occurs more commonly through initial penetration in localized areas rather than widespread airborne transmission, sensors may be installed on the filter 260 where air flows in, and when viruses are detected by the sensors, the injector 250 may immediately target and spray toward the sensor direction to instantly destroy viruses.

The filter 260 may be formed in one area constituting the breeding space 230. The filter 260 may be installed in an upper area, but the installation height is not limited to one example and may be installed at various heights and positions. The filter 260 may perform a barrier role to prevent some viruses that could not be destroyed by disinfectants from penetrating.

The breeding space 230 may be installed with separate inner walls, frames, exterior materials, interior materials, iron bars, etc. to bound the space where animals can be raised in all directions inside the housing 210. The breeding space 230 can define an independent space within the housing 210 through such structures and allow animals to be raised within it. The breeding space 230 is configured to be sealed except for the space where the filter 260 is installed and may be configured to be spaced apart from the opening/closing ports 220a, 220b.

Meanwhile, referring to FIG. 5, outside air introduced through the first opening/closing port 220a is not directly introduced into the breeding space 230, but may be introduced into the breeding space 230 via the first separation space 231 and second separation space 232. The first separation space 231 is defined as the space between the first opening/closing port 220a and the air conditioning device 240, and the second separation space 232 may be defined as the space between the air conditioning device 240 and the filter 260. Outside air is introduced into the first separation space 231, cooled or heated outside air is introduced into the second separation space 232 by the heating/cooling air conditioning device 240 between the first separation space and the second separation space 232, and viruses in outside air in the second separation space 232 may be removed by disinfectant sprayed from the filter 260 and injector 250. Here, two separation spaces are described, but this is an example, and it may

be configured with one separation space or more than two separation spaces. Separation spaces may be separated from neighboring spaces by sealed walls and can perform the role of virus blocking by themselves. Cases may occur where viruses stick to walls, etc. while passing through separation spaces, and by preventing animals from directly contacting outside air, virus exposure risk can be reduced. Each separation space may be composed of sealed walls except for passages connecting to neighboring spaces. Additionally, the number of separation spaces at the position where outside air is introduced may differ from the number of separation spaces at the position where outside air is discharged. This is because when outside air exits, it has already passed through the breeding space 230, so rapid discharge is more important, and there is no need to create hurdles by having multiple separation spaces.

The sensor unit 270 includes a plurality of sensors installed inside the smart livestock housing system 200. The plurality of sensors may be composed of different types of sensors. For example, it may be composed of various sensors such as temperature sensors, humidity sensors, atmospheric pressure measurement sensors, vibration sensors, carbon dioxide concentration measurement sensors, virus detection sensors, etc. Sensors may be installed at various locations. They may be installed in the breeding space 230, first separation space 231, second separation space 232, etc., and may also be installed on the outer wall of the housing 210. Sensors installed in the breeding space 230 may be arranged as sensors to check uniformity of temperature and humidity and presence or absence of viruses, and may be installed in plurality on the floor, sides, and ceiling respectively. The sensor unit 270 can transmit measured information to the server 100 and operate according to commands from the server 100.

Among the sensors, virus detection sensors may be configured in the form of a substrate combined with aptamers and reaction solutions, configured to change color when viruses combine with the aptamers and reaction solutions. Specifically, virus detection sensors may (I) measure virus or bacterial density information inside and outside livestock housing for highly pathogenic diseases by livestock type (e.g., avian influenza, foot-and-mouth disease, swine fever, salmonella, etc.). It may additionally include virus detection sensors using aptamers that specifically bind to influenza viruses. Aptamers may be labeled by being attached to detection substances. Any substance may be used as the detection substance as long as it can confirm whether aptamers have bound to influenza viruses. When detection substances are tagged to aptamers, it is preferable that the detection substances are attached so as not to affect specificity or selectivity for targets in the aptamers, and for this purpose, they may be provided linked (covalently bonded or crosslinked) to the aptamers.

Virus detection sensors may include substrates on which two or more aptamers are immobilized to detect chemical reactions of viruses. At this time, the substrate may be other materials on which aptamers can be immobilized, for example, substrates, resins, plates (e.g., multiwell plates), filters, cartridges, columns or porous materials, or those used in DNA chips or protein chips, nickel-PTFE (polytetrafluoroethylene) substrates or glass substrates, apatite substrates, silicon substrates, gold, silver or alumina substrates, etc., or those substrates coated with polymers, etc.

Virus detection sensors may be configured with aptamers spotted on bio chips (or substrates) together with fluorescent materials, coloring materials, or antibodies, and at this time, outside air flows in and may react with trace amounts of influenza viruses contained in the outside air. For example, when fluorescent materials are used as labeling materials, luminescence or color changes occur in the presence of target substances, and target substances can be detected by measuring this. Alternatively, virus detection sensors may scan wells that have reacted through image scanners capable of detecting fluorescent dyes to confirm detection of target substances.

Virus detection sensors are attached to the outside of livestock housing, intake filter parts of air purification chambers, upper and lower layers inside farms (front, middle, rear), outside air filter parts, and inside circulators respectively, and when viruses are detected, information may be transmitted to the user terminal 300 through chemical reactions.

As an additional embodiment, virus detection sensors may be manufactured in forms such as bottles, tubs, sachets, envelopes, ampoules, etc., and may be manufactured partially or entirely using plastic, glass, paper, foil, or wax. When configured in container form, containers may include completely or partially separable stoppers that are part of the container or may be attached to the container by mechanical, adhesive, or other means; and stoppers through which contents can be accessed by syringes.

Referring to FIG. 5, it can be confirmed that outside air introduced into the smart livestock housing system 200 is introduced into the breeding space 230 via the first separation space 231 and second separation space 232, then exits or circulates again. In this process, viruses existing outside can be filtered primarily through each separation space, secondarily destroyed by disinfectants sprayed from the injector 250, and some viruses that still survive are removed by the filter 260, so animals in the breeding space 230 can be safely protected from virus infection.

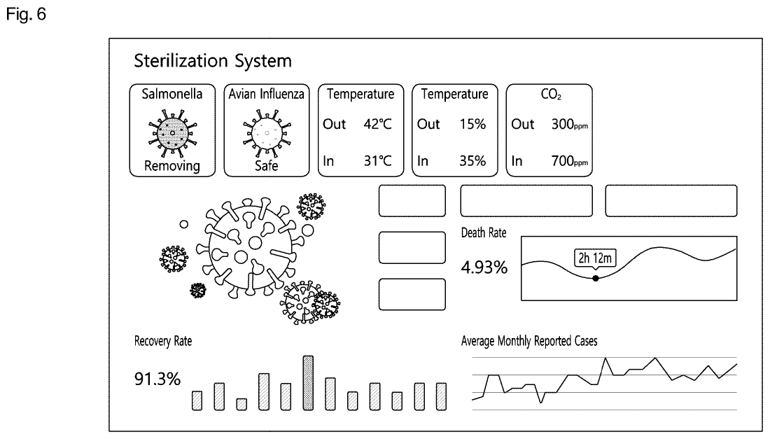

FIG. 6 is an example of a user interface provided to the user terminal 300 according to one embodiment of the present invention. Status information and control information of such smart livestock housing system 200 may be provided to the user terminal 300, and as shown in FIG. 6, information about the presence or absence of viruses such as salmonella and influenza bacteria may be presented in the user interface. In addition, current information, past information, and time-series data regarding temperature, humidity, carbon dioxide concentration, etc. may be provided. This is only an example, and user interfaces may be presented in various forms.

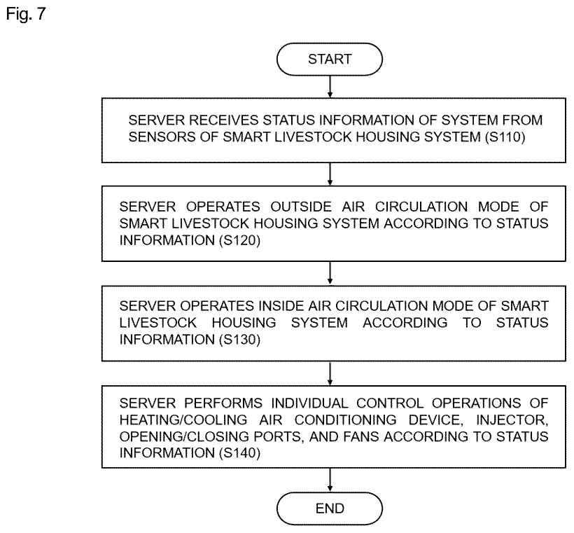

Referring to FIG. 7, the server 100 may first receive status information of the system from the sensor unit 270 of the smart livestock housing system 200 (S110).

The server 100 may operate the outside air circulation mode of the smart livestock housing system 200 according to the status information (S120). For example, when the risk of virus presence outside is low and it is determined that outside air inflow is necessary for a comfortable environment for animals, the injector 250 may be operated at appropriate intervals while allowing outside air to flow into the breeding space 230.

In this case, the smart livestock housing system 200 may operate through: operating a plurality of opening/closing ports 220a, 220b to introduce outside air into the interior; performing an operation to remove viruses by spraying disinfectant into outside air through the injector 250; and allowing outside air that has passed through the injector 250 to be introduced into the interior of the breeding space 230 formed to bound the space where animals are raised in all directions through the filter 260.

The server 100 may also operate the inside air circulation mode of the smart livestock housing system 200 according to the status information (S130). When it is determined that there is a high virus risk outside, it may operate in inside air circulation mode. In this case, while sealing the opening/closing ports 220a, 220b, inside air is continuously circulated, and the injector 250 may also be operated at appropriate intervals so that viruses in the circulating inside air are completely destroyed.

Additionally, the server 100 may perform individual control operations of the heating/cooling air conditioning device 240, injector 250, opening/closing ports 220a, 220b, and fans 230a-230c may be controlled differently.

Existing virus collection methods manually replace aptamers and reaction materials of collection devices periodically and are designed to react positively when virus contamination reaches a certain level or higher, so small amounts of viruses cannot be detected and immediate response is difficult.

To solve this, as an additional embodiment, when individual IoT devices are attached to each farm and their status information is collected by different external servers or the server 100 of the present invention, the server 100 may collect regional data on virus and bacteria detection. This enables regionalized data collection. Subsequently, the server 100 may generate solutions that can immediately treat farms in real time based on collected disease information. And based on collected data, it can analyze the presence or absence of specific disease occurrence and infection risk. And analysis results may be provided to the user terminal 300 of each farm. Through this, an autonomous control system capable of immediate on-site response through data analysis when virus detection and abnormal signs are detected based on collected real-time information about viruses and inhaled air conditions can be established.

At this time, the server 100 may set different spraying cycles of injectors 250 to be controlled for each farm and ventilation cycles through outside air mode operation according to whether infection levels are high or low based on regional disease area density and disease and infection status of users' livestock housing based on collected disease information, and may control by transmitting differently set results to the smart livestock housing system 200 of each farm. Furthermore, it may differently control the disease management level (disinfectant spraying cycle or outside air circulation cycle, etc.) of users' livestock housing even when there are no diseases in surrounding livestock housing due to seasonal factors.

As an additional embodiment, the server 100 may utilize video analysis (VS, Video Analysis) techniques to analyze behavioral patterns of individual animals and, when behavior deviating from normal behavioral patterns accumulated in existing databases is detected, identify it as abnormal signs and perform diagnosis on whether diseases have occurred, whether there is a possibility of disease occurrence, what kind of disease it is, etc. At this time, various acoustic information can be commonly used for analysis of animal diseases or abnormal behavior patterns. Among them, acoustic information including breathing and coughing sounds of objects can be collected to primarily determine the presence or absence of diseases, and secondary judgment can be made through abnormal sign analysis using video analysis techniques to estimate the presence or absence of diseases and disease types.

Additionally, as an additional embodiment, the server 100 may calculate the possibility of future avian influenza (disease) occurrence based on regional differences in avian influenza (disease) occurrence frequency and current regional avian influenza occurrence status, and determine what measures the livestock housing should take based on artificial intelligence.

Regional differences in disease occurrence frequency may occur due to animal population density in the breeding space 230, bird migration routes (birds migrate seasonally and can pass through various regions, spreading viruses in this process), environmental factors (for example, areas with high humidity or abundant water may have higher disease occurrence frequency because viruses can survive longer), livestock management level (facilities with well-maintained hygiene have low virus transmission rates), quarantine policies (regions with strict quarantine and inspection have low avian influenza occurrence frequency), etc. Therefore, even if avian influenza shows signs of nationwide epidemic, if it is judged that avian influenza occurrence frequency will be low in the livestock housing according to the factors explained above, it would be desirable to control in the direction of properly managing the livestock housing system 200 while providing appropriate ventilation to poultry in the livestock housing. Therefore, based on this, data on the number of avian influenza (disease) occurrences by period for each region, bird population density, seasonal bird migration routes, environmental factors (temperature, humidity), regional hygiene management levels, regional quarantine frequency, etc. may be collected from external servers, and avian influenza occurrence probability in the region may be calculated through artificial intelligence models. That is, when learning is performed by setting the collected data as input values and setting the output value as the number of livestock housing where avian influenza actually occurred, when learning is completed, avian influenza occurrence probability in the region (number of predicted infection-possible livestock housing compared to total livestock housing in the region) can be calculated. At this time, artificial intelligence models may use supervised learning models (ex. KNN, LINEAR REGRESSION) or reinforcement learning models (ex. DQN, A3C), and based on this, methods for controlling IoT devices (opening/closing ports 220a, 220b, fans 230a-230c, injector 250, and air conditioning device 240 to be more frequent than usual. When avian influenza occurrence probability is calculated to be low, operations corresponding to outside air circulation may be commanded to IoT devices while controlling the air conditioning device 240 to operate energy-efficiently so that outside air blows in frequently for more frequent temperature control by outside air, but controlling by reducing the operation cycle of the injector 250 compared to usual.

One embodiment of the present invention may also be implemented in the form of a recording medium including instructions executable by a computer, such as program modules executed by a computer. Computer-readable media may be any available media that can be accessed by a computer and includes both volatile and nonvolatile media, removable and non-removable media. Additionally, computer-readable media may include all computer storage media. Computer storage media includes both volatile and nonvolatile, removable and non-removable media implemented in any method or technology for storage of information such as computer-readable instructions, data structures, program modules, or other data.

An embodiment of the present disclosure may also be implemented in the form of a recording medium including instructions executable by a computer, such as a program module executed by the computer. Computer-readable media may be any available media that may be accessed by a computer and include both volatile and nonvolatile media and removable and non-removable media. In addition, the computer-readable media may include all computer storage media. The computer storage media includes both volatile and nonvolatile media and removable and non-removable media implemented by any method or technology of storing information, such as a computer readable instruction, a data structure, a program module, and other data.

Although the method and system according to the present disclosure are described with reference to specific embodiments, some or all of their components or operations may be implemented by using a computer system having a general-purpose hardware architecture.

The above descriptions on the present disclosure are for illustration, and those skilled in the art to which the present disclosure pertains may understand that the descriptions may be easily modified into other specific forms without changing the technical idea or essential features of the present disclosure. Therefore, it should be understood that the embodiments described above are illustrative in all respects and not restrictive. For example, each component described as a single type may be implemented in a dispersed form, and likewise components described as distributed may be implemented in a combined form.

The scope of the present disclosure is indicated by the following claims rather than the above detailed description, and all changes or modifications derived from the meaning and scope of the claims and their equivalents should be interpreted as being included in the scope of the present disclosure.

### DESCRIPTION OF SYMBOLS

100: server 200: smart livestock housing system
300: user terminal.

What is claimed is:

1. An active smart livestock housing system capable of disease prevention, comprising:
a housing formed to have an internal space for raising animals;
a plurality of opening/closing ports formed on one side and another side of the housing, respectively, to control inflow of outside air into an interior of the housing;
a breeding space formed to bound the space where animals are raised in all directions inside the housing;
at least one filter formed in one area of the breeding space; and
an injector located between the opening/closing ports and the filter, operating to remove viruses by spraying disinfectant into the outside air entering from the opening/closing ports.

2. The system of claim 1, further comprising:
a first sensor unit installed on an outer wall of the housing and a second sensor unit installed inside the housing,
wherein the first sensor unit and the second sensor unit each measure at least one of temperature, humidity, carbon dioxide concentration, and virus detection presence, and when the second sensor unit is installed inside the breeding space, a plurality of the second sensor units are installed in plurality on a floor, sides, and ceiling.

3. The system of claim 2, wherein each of the first sensor unit and the second sensor unit is configured in the form of a substrate combined with aptamers and reaction solutions, configured to change its color when viruses combine with the aptamers and the reaction solutions.

4. The system of claim 1, further comprising: a heating/cooling air conditioning device disposed between at least one of the opening/closing ports and the filter, wherein the heating/cooling air conditioning device performs cooling or heating operations when outside air is introduced into the interior of the housing, controlling at least one of temperature and humidity of air introduced into the breeding space.

5. The system of claim 1, further comprising: a fan installed on an inner side of the housing of one of the opening/closing ports, wherein the fan is configured as an intake fan or exhaust fan to control speed at which outside air blows out or comes in.

6. The system of claim 1, wherein the breeding space is configured to be sealed except for the space where the filter is installed and is configured to be spaced apart from the opening/closing ports,
a space between the breeding space and the opening/closing ports includes at least one of a first separation space and a second separation space,
number of separation spaces at a position where outside air is introduced differs from number of separation spaces at the position where outside air blows out,
the first separation space and the second separation space are separated by sealed walls,
outside air is introduced into the first separation space,
cooled or heated outside air is introduced into the second separation space by a heating/cooling air conditioning device between the first separation space and the second separation space, and
viruses in the outside air introduced into the second separation space are removed by disinfectant sprayed from the filter and the injector.

7. The system of claim 1, further comprising: a sensor installed on the filter to detect the presence or absence of viruses, wherein when the sensor detects viruses, the injector operates to target an angle toward a direction in which the sensor is located and then performs spraying operations to destroy the viruses.

8. The system of claim 7, wherein: each opening/closing port is installed on different surfaces spaced apart from each other within the housing to discharge outside air introduced from one opening/closing port, and the filter and the injector are located adjacent to each opening/closing port to perform virus removal functions.

9. The system of claim 1, further comprising: a server that monitors status of devices including first and second sensor units, the opening/closing ports, a heating/cooling air conditioning device, the injector, and a fan, and controls their operation.
